(19) United States
(12) Patent Application Publication
(10) Pub. No.: US 2025/0195159 A1
(43) Pub. Date: Jun. 19, 2025

(54) Hybrid Optical-Inertial Bone Tracking

(71) Applicant: MAKO Surgical Corp., Weston, FL (US)

(72) Inventors: Sai Manoj Prakhya, Freiburg (DE); Lara Rebecca Klaiber, Eppingen (DE); Sascha Schlechtweg, Baden-Württemberg (DE); Jonathan Mark Morgan, Biscayne Park, FL (US)

(73) Assignee: MAKO Surgical Corp., Weston, FL (US)

(21) Appl. No.: 18/985,115

(22) Filed: Dec. 18, 2024

Related U.S. Application Data
(60) Provisional application No. 63/611,298, filed on Dec. 18, 2023.

(51) Int. Cl. A61B 34/30; A61B 17/70; A61B 34/20; A61B 90/00

(52) U.S. Cl. CPC A61B 34/30; A61B 90/39; A61B 17/7074; A61B 2034/2048; A61B 2034/2055; A61B 2090/067; A61B 2090/3916; A61B 2090/3937; A61B 2090/3991

(57) ABSTRACT

First and second tracker assemblies each having a single optical marker are secured to a bone, with the first tracker assembly also including a motion sensor. A localizer optically tracks the optical marker positions, and a pose of a tracker coordinate system (TCS) associated with the tracker assemblies according to three positional DOF and two rotational DOF is determined from the tracked marker positions. Measurements from the motion sensor are utilized to determine an angle of inclination with respect to gravity, and an orientation of the TCS according to a further rotational DOF defined about a virtual line extending between the optical markers is determined from the angle of inclination. A pose of the bone according to six DOF is then determined from the determined pose of the TCS according to three positional and two rotational DOF and the orientation of the TCS according to the further rotational DOF.

### CROSS-REFERENCE TO RELATED APPLICATIONS

The subject application claims priority to and all the benefits of U.S. Provisional Patent App. No. 63/611,298, filed Dec. 18, 2023, the entire contents of which are hereby incorporated by reference.

### BACKGROUND

Surgical systems typically employ navigated tracking to detect the pose of a bone undergoing surgery. Depending on the surgery, the tracked bone can be part of the knee joint, hip joint, should joint, or spine, for example. The human spine is characterized by a specific curvature allowing the spine to absorb impacts, which can be disturbed by degenerative changes or spinal deformities. Minimally invasive interbody fusion is an established treatment option for degenerative spine pathologies, and generally involves inserting an implant (spacer, graft, cages) into the intervertebral disc. Intervertebral movement during such procedure can affect the outcome if not taken under consideration. Computer assisted surgeries with surgical navigation have the advantage of tracking intraoperative movement of patient anatomy in six degrees of freedom (DOF) by cooperating with trackers attached to the bone. However, since vertebra are relatively small and close together, especially in the cranial-caudal direction, attaching conventional 6 DOF trackers to individual vertebra is difficult, especially in the minimally invasive context. The placement of the implant and the adjustment of vertebra within the spine's curvature are thus typically realized without surgical navigation tracking and based on the surgeon's experience and skill.

### SUMMARY

This Summary introduces a selection of concepts in a simplified form that are further described below in the Detailed Description below. This Summary is not intended to limit the scope of the claimed subject matter nor identify key features or essential features of the claimed subject matter.

A first aspect includes a hybrid tracker for tracking an object of interest in a medical procedure, such as a patient bone, instrument, or robotic manipulator, in six degrees of freedom. The hybrid tracker includes two optical markers configured to be coupled to the object and detectable by a localizer. The tracker also includes a motion sensor moveable with at least one of the two optical markers and configured to produce measurements indicative of an angle of inclination with respect to gravity.

A second aspect includes a method for tracking an object of interest in a medical procedure, such as a patient bone, instrument, or robotic manipulator, in six degrees of freedom, such as using the tracker of the first aspect. The method includes tracking optically, with a localizer, positions of the optical markers in a known coordinate system, such as a coordinate system of the localizer. The method also includes determining, by one or more controllers, a pose of the tracker, or more particularly of a tracker coordinate system associated with the tracker, in the known coordinate system, such as according to three positional degrees of freedom and two rotational degrees of freedom, based on the tracked positions of the optical markers. The method also includes utilizing, by the one or more controllers, measurements from the motion sensor to determine an angle of inclination with respect to gravity. The method also includes determining, by the one or more controllers, an orientation of the tracker, or more particularly of the tracker coordinate system, according to a further rotational degree of freedom, such as defined about a virtual line extending between the optical markers, based on the angle of inclination. The method also includes determining, by the one or more controllers, a pose of the object in the known coordinate system according to six degrees of freedom based on the pose of the tracker, or more particularly of the tracker coordinate system, determined based on the tracked positions of the optical markers, and the orientation of the tracker, or more particularly of the tracker coordinate system, determined based on the angle of inclination.

Other aspects include corresponding computer systems, apparatus, and computer programs recorded on one or more computer storage devices, each configured to perform the actions of the above method. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A third aspect includes a surgical navigation system for tracking an object of interest in a medical procedure, such as a patient bone, instrument, or robotic manipulator, in six degrees of freedom, such as using the tracker of the first aspect. The system includes a localizer configured to optically track positions of the optical markers of the tracker in a known coordinate system, such as a coordinate system of the localizer. The system also includes one or more controllers configured to: determine a pose of the tracker, or more particularly of a tracker coordinate system associated with the tracker, in the known coordinate system, such as according to three positional degrees of freedom and two rotational degrees of freedom, based on the tracked positions of the optical markers; utilize measurements from the motion sensor to determine an angle of inclination with respect to gravity; and determine an orientation of the tracker, or more particularly of the tracker coordinate system, according to a further rotational degree of freedom, such as defined about a virtual line extending between the optical markers, based on the angle of inclination. The one or more controllers are also configured to determine a pose of the object in the known coordinate system according to six degrees of freedom based on the pose of the tracker, or more particularly of the tracker coordinate system, determined based on the tracked positions of the optical markers, and the orientation of the tracker, or more particularly of the tracker coordinate system, determined based on the angle of inclination.

A fourth aspect includes a surgical system including a robotic manipulator and the surgical system of the third aspect. The one or more controllers are configured to control movement of the robotic manipulator relative to the object during the medical procedure based on the determined pose of the object in six degrees of freedom.

A fifth aspect includes a surgical navigation system comprising: a tracker assembly being rigidly attached to a bone and comprising at least one optical marker; a motion sensor being rigidly attached to the bone and being configured to measure an angle of inclination with respect to gravity; a localizer configured to optically detect a pose of the at least one optical marker; and one or more controllers coupled to the localizer and motion sensor being configured to: receive the measured angle of inclination; establish a baseline relationship between the at least one optical marker and the motion sensor by combining the detected pose of the at least one optical marker and the measured angle of inclination; monitor a measured relationship between the detected pose of the at least one optical marker and the measured angle of inclination; and detect a deviation between the measured relationship and the baseline relationship to identify that the at least one optical marker has moved relative to the bone.

A sixth aspect includes a method of operating the surgical navigation system of the fifth aspect.

A seventh aspect includes a surgical navigation system comprising: a tracker assembly being rigidly attached to a bone and comprising: at least one optical marker, and a tracker motion sensor configured to measure a first angle of inclination with respect to gravity; a tracker observation sensor being rigidly attached to the bone and configured to measure a second angle of inclination with respect to gravity; a localizer configured to optically detect a pose of the at least one optical marker; one or more controllers coupled to the localizer, the tracker motion sensor, and the tracker observation sensor and being configured to: receive the measured first and second angles of inclination; establish a baseline relationship between the tracker assembly and the tracker observation sensor by combining the measured first and second angles of inclination; track the measured first and second angles of inclination to monitor a measured relationship between the tracker assembly and the tracker observation sensor; and detect a deviation between the measured relationship and the baseline relationship to identify that the at least one optical marker has moved relative to the bone.

An eight aspect includes a method of operating the surgical navigation system of the seventh aspect.

A ninth aspect includes a tracker observation device configured to monitor a tracker assembly mounted to a bone at a first location, the tracker observation device comprising: a body: an attachment coupled to the body and being configured to be mounted to the bone at a second location spaced apart and different from the first location; a motion sensor coupled to the body and being configured to measure an angle of inclination with respect to gravity; and a communication device coupled to the body and being configured to remotely transmit the measured angle of inclination.

Any of the above aspects may be combined in part or in whole. The above aspects may incorporate any one or more of the following implementations, in part or in whole:

In some implementations, the tracker includes only two optical markers. In some implementations, the tracker includes a first tracker assembly having one of the optical markers and includes a second tracker assembly having another one of the optical markers. In accordance with the previous implementation, each tracker assembly may thus have only one optical marker. The first tracker assembly is coupleable to the object at a first location, and the second tracker assembly is coupleable to the object at a second location different from the location. The first tracker assembly also includes the motion sensor.

In some implementations, the object being tracked is a bone, and the first and second tracker assemblies are removably coupleable to first and second pedicle screws inserted into the bone, respectively. The bone can be more vertebra, a femur, a tibia, a scapula, a humerus, a pelvis, a skull, or the like.

In some implementations, the motion sensor is configured to measure movement of to an axis substantially perpendicular with a virtual line extending between the optical markers of the tracker. The angle of inclination with respect to gravity used to determine the orientation of the tracker according to the further rotational degree of freedom may thus be defined as an angle of inclination of such axis with respect to gravity.

In some implementations, the motion sensor is defined as an accelerometer. In some implementations, the tracker, including the first tracker assembly and the second tracker assembly when implemented, does not include a magnetometer, and/or does not include a gyroscope.

In some implementations, an initial pose of the tracker, or more particularly of the tracker coordinate system, is determined in the known coordinate system according to six degrees of freedom. In some implementations, measurements from the motion sensor that correspond to the initial pose are utilized to obtain an initial angle of inclination relative to gravity. In some implementations, the pose of the tracker, or more particularly of the tracker coordinate system, in the known coordinate system according to three positional degrees of freedom and two rotational degrees of freedom is determined based on the tracked positions of the optical markers and the initial pose. In some implementations, the orientation of the tracker, or more particularly of the tracker coordinate system, according to the further rotational degree of freedom is determined based on the determined angle of inclination and the initial angle of inclination.

In some implementations, a first vector connecting the optical markers of the first and second tracker assemblies is calculated in the known based on the initial pose; a second vector connecting the optical markers of the first and second tracker assemblies in the know coordinate system is calculated based on the tracked positions of the optical markers; a rotational matrix is calculated from the first and second vectors; and the pose of the tracker, or more particularly of the tracker coordinate system, in the known coordinate system according to the three positional and two rotational degrees of freedom is determined based on the rotational matrix.

In some implementations, a rotational matrix is calculated from the initial and determined angles of inclination; and the orientation of the tracker, or more particularly of the tracker coordinate system, according to the further rotational degree of freedom is determined based on the rotational matrix calculated from the initial and determined angles of inclination.

In some implementations, the tracker, such as at least one of the first and second tracker assemblies, includes a feature for temporary contact by a further tracker assembly including at least one optical marker detectable by the localizer for determining the initial pose of the tracker, or more particularly of the tracker coordinate system, in the known coordinate system according to six degrees of freedom.

In some implementations, a second motion sensor is moveable with the localizer. Measurements from the second motion sensor that correspond to the initial pose are utilized to obtain a second initial angle of inclination with respect to gravity. In some implementations, the initial pose of the tracker, or more particularly of the tracker coordinate system, in the known coordinate system according to six degrees of freedom is determined based on the tracked positions of the optical markers corresponding to the initial pose, the initial of inclination with respect to gravity indicated by the motion sensor of the tracker, and the second initial of inclination with respect to gravity indicated by the second motion sensor. In some implementations, the orientation of the tracker, or more particularly of the tracker coordinate system, according to the further rotational degree of freedom is determined based on the determined angle of inclination, the initial angle of inclination, and the second initial angle of inclination.

In some implementations, such as when the axis the movement of which is measured by the motion sensor is not substantially perpendicular to the virtual line extending between the optical markers, an angle between the axis and a plane normal to the virtual line extending between the optical markers is determined; and the orientation of the tracker, or more particularly the tracker coordinate system, according to the further rotational degree of freedom defined about the virtual line extending between the optical markers is determined based on the determined angle of inclination and the determined angle between the axis and the plane. In some implementations, measurements from the motion sensor are monitored to determine whether a tracker assembly has moved relative to the bone.

The motion sensor can be coupled to the tracker assembly. The tracker assembly can include an attachment portion that is coupled to the bone, a stem extending from the attachment portion, and a tracking head being coupled to the stem and supporting the at least optical marker. The motion sensor can be coupled to any component of the tracker assembly. The tracker assembly can be attached to the bone at a first location. The motion sensor can be rigidly attached to the bone at a second location spaced apart and different from the first location. A second tracker assembly can be rigidly attached to the bone at the second location. The second tracker assembly can include at least one optical marker and the motion sensor is coupled to the second tracker assembly. The motion sensor can be coupled to a tracker observation device at the second location.

The tracker observation sensor is coupled to the tracker assembly at the first location. The tracker observation sensor can be coupled to any component of the tracker assembly. The tracker observation sensor can be coupled to a different component of the tracker assembly than the component to which the motion sensor is attached. The tracker observation sensor can be rigidly attached to the bone at a second location spaced apart and different from the first location. The second tracker assembly can include at least one optical marker and the tracker observation sensor is coupled to the second tracker assembly. The tracker observation sensor can be coupled to a tracker observation device at the second location. The one or more controllers can establish the baseline relationship between the tracker assembly and the tracker observation sensor by further combining the measured first and second angles of inclination with the tracked pose of the at least one optical marker of the tracker assembly. The one or more controllers are configured to generate feedback in response to detection of the deviation between the measured relationship and the baseline relationship. Implementations of the described techniques may include hardware, a method or process, or computer software on a computer-accessible medium.

### BRIEF DESCRIPTION OF THE DRAWINGS

FIG. 1 illustrates a surgical system for performing a spinal surgery, the surgical system including a robotic manipulator for guiding an end effector to manipulate spinal tissue and a navigation system for tracking a vertebra during the procedure.

FIG. 2 illustrates a hybrid tracker for tracking an object that may be incorporated into the navigation system of FIG. 1.

FIG. 3 is an enlarged view of the hybrid tracker of FIG. 2.

FIG. 4 illustrates components that may be incorporated into the surgical system of FIG. 1.

FIG. 5 illustrates a method for tracking an object using the hybrid tracker of FIG. 2.

FIG. 6 illustrates a further method for tracking an object using the hybrid tracker of FIG. 2.

### DETAILED DESCRIPTION

The following description provides exemplary implementations with reference to the drawings. The same or similar reference numerals will be used to denote the same or similar structural features.

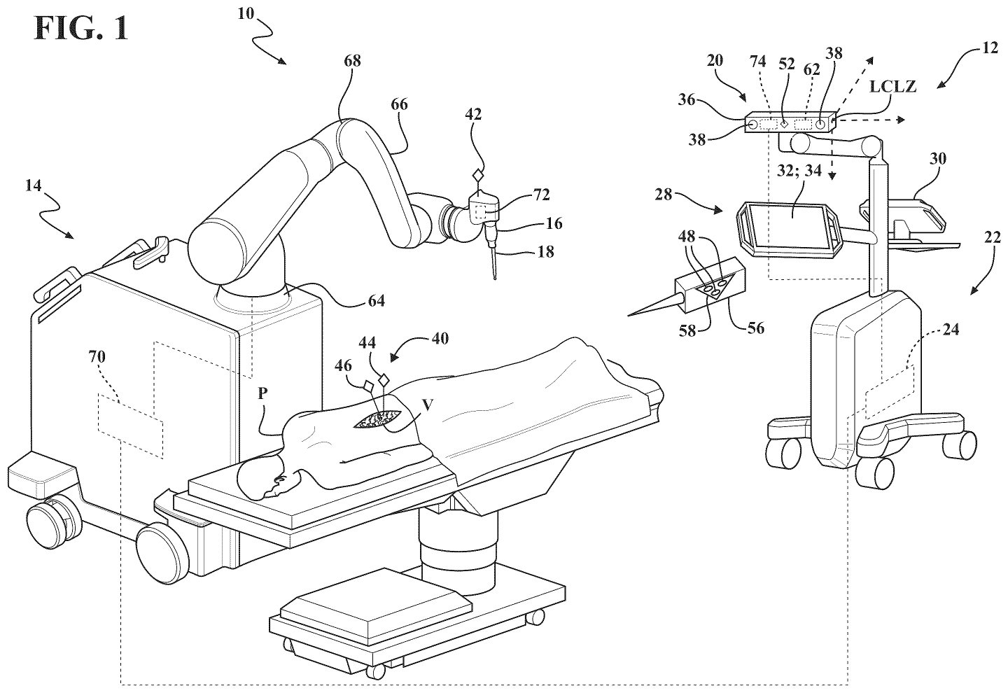

FIG. 1 illustrates a surgical system 10 for treating an anatomy (i.e., target site) of a patient P. The surgical system 10 may be located in a surgical setting such as an operating room of a medical facility. The surgical system 10 may include a surgical navigation system 12 and a robotic manipulator 14.

The robotic manipulator 14 may be coupled to a surgical instrument 16 and may be configured to maneuver the surgical instrument 16 to treat a target volume of patient tissue, such as at the direction of a surgeon and/or the surgical navigation system 12. For example, the surgical navigation system 12 may cause the robotic manipulator 14 to maneuver the surgical instrument 16 to manipulate and/or remove the target volume of patient tissue while avoiding other objects adjacent the target volume in the surgical workspace, such as other medical tools and adjacent anatomical structures desired to be avoided. Additionally, or alternatively, the surgeon may manually hold and maneuver the surgical instrument 16 while receiving guidance from the surgical navigation system 12 and/or from the robotic manipulator 14. For instance, the robotic manipulator 14 may be configured to provide haptic feedback and/or to constrain movement of the surgical instrument 16 as the surgical instrument 16 is moved by the surgeon relative to the target volume to avoid adjacent objects.

The surgical instrument 16 may be configured to manipulate and/or remove tissue at the target site, and/or to insert an implant into tissue at the target site, which may include soft tissue and/or hard tissue such as bone. For example, and without limitation, the surgical instrument 16 may be a burring instrument, an electrosurgical instrument, an ultrasonic instrument, a reamer, an impactor, or a sagittal saw. As described in more detail below, the surgical instrument 16 may also be an instrument specific to performing a spinal surgery, such as a cobb, box chisel, curette, cutter, dilator, or inserter.

The surgical navigation system 12 may be configured to track the pose (i.e., location and orientation) of objects of interest within the surgical workspace using tracker-based localization. The tracked objects may include, but are not limited to, anatomical structures of the patient, surgical instruments such as the surgical instrument 16, and anatomical structures of surgical personnel such as the surgeon's hand or fingers. The tracked anatomical structures of the patient may include soft tissue such as ligaments, muscle, and skin, and/or may include hard tissue such as bone. The tracked surgical instruments may include retractors, cutting tools, inserters, implants, and waste management devices used during the surgical procedure.

### I. Hybrid Optical-Inertial Bone Tracking

Each object of interest may be affixed to a tracker that is configured to transmit light to the surgical navigation system 12. At least one of the affixed trackers may also include a motion sensor configured to generate motion measurement data indicative of an angle of inclination with respect to gravity. For instance, in the example illustrated in FIG. 1, the surgical system 10 is being used to perform a spinal procedure on a patient P, such as a minimally invasive interbody fusion in which an implant (e.g., spacer, graft, cage) is inserted into the intervertebral disc space. To support an optimal positioning of the implant and an optimal alignment of vertebra V according to a target spine curvature, the surgical navigation system 12 may be configured to continuously track the pose of individual vertebra V during the spinal procedure in 6 DOF. One possible approach to providing such tracking would be to mount a 6 DOF optical tracker including at least three optical markers on a spine clamp that is attached to spinous processes of vertebra V. However, in view of the relatively small size and close arrangement of the vertebra V, such approach would crowd the surgical site and make performing the procedure as a minimally invasive surgery difficult.

The present disclosure describes implementations of a small, lightweight, minimally invasive 6 DOF tracker optimized for tracking individual vertebra V. Such tracker may include two, or more particularly only two, optical markers and a motion sensor configured to be coupled to the vertebra V. The optical markers may emit light for determining a pose of the tracker in 5 DOF, and the motion sensor may generate data for determining an orientation of the tracker in a further rotational DOF.

Although largely described in the context of tracking vertebral bodies, it will be understood that the tracking techniques and advantages described herein are not limited only to vertebral bodies, and may be utilized for tracking any type of bone structure of the patient P including without limitation a femur, a tibia, a scapula, a humerus, pelvic bone, ribs, or the skull. Moreover, the tracker can be used in any appropriate surgical procedure where tracking of one or more bones. Such surgical procedures include but are not limited to: spinal procedures, partial or total knee arthroplasty, shoulder arthroplasty, hip arthroplasty, cranial procedures, or the like.

The surgical navigation system 12 may be configured to detect the light signals emitted from a given tracker by imaging the tracker, and to receive motion measurement data from a given tracker via a wired or wireless data connection with the tracker. Alternatively, a given tracker with a motion sensor may be configured to encode the motion measurement data in the light signals emitted from the optical markers of the tracker, such as by setting the intensity, frequency, and or pattern of the emitted light according to the motion measurement data. The surgical navigation system 12 may then be configured to determine the poses of the trackers in a known coordinate system according to 6 DOF based on the imaging and the motion data, and thereafter determine the poses of the objects to which the trackers are affixed in the known coordinate system according to 6 DOF based on the determined poses of the trackers and predetermined positional relationships between the objects and trackers.

Responsive to determining the poses of objects of interest according to 6 DOF, the surgical navigation system 12 may display the relative poses of the tracked objects to aid the surgeon. The surgical navigation system 12 may also control and/or constrain movement of the robotic manipulator 14 and/or surgical instrument 16 based on virtual boundaries associated with the tracked objects. For example, the surgical navigation system 12 may identify a target volume of patient tissue to be treated and potential obstacles in the surgical workspace based on the tracked objects. The surgical navigation system 12 may then restrict a surgical tool (e.g., an end effector 18 of the surgical instrument 16) from contacting anything beyond the target volume of patient tissue to be treated, improving surgical accuracy. The surgical navigation system 12 may also eliminate damage to surgical instruments caused by unintended contact with other objects, which may result in undesired debris at the target site.

Still referring to FIG. 1, the surgical navigation system 12 may include a localizer camera 20 and a navigation cart assembly 22. The navigation cart assembly 22 may house a navigation controller 24 configured to implement the functions, features, and processes of the surgical navigation system 12 described herein. For instance, the navigation controller 24 may be configured to convert optical-based image data received from the localizer camera 20 into object pose data indicative of the poses of the tracked objects in the surgical workspace, and to control operation of the robotic manipulator 14 based on the indicated poses as described herein.

The navigation controller 24 may be in operative communication with a user interface 28 of the surgical navigation system 12. The user interface 28 may facilitate user interaction with the surgical navigation system 12 and navigation controller 24. For example, the user interface 28 may include one or more output devices that provide information to a user, such as from the navigation controller 24. The output devices may include a display 30 adapted to be situated outside of a sterile field including the surgical workspace and may include a display 32 adapted to be situated inside the sterile field. The displays 30, 32 may be adjustably mounted to the navigation cart assembly 22. The user interface 28 may also include one or more input devices that enable user-input to the surgical navigation system 12. The input devices may include a keyboard, mouse, and/or touch screen 34 that can be interacted with by a user to input surgical parameters to and control aspects of the navigation controller 24. The input devices may also include a microphone that enables user input through voice-recognition technology.

The localizer camera 20 may be configured to facilitate the identification of the poses of the tracked objects in the surgical workspace relative to a known coordinate system, such as a localizer coordinate system LCLZ of the localizer camera 20, by generating image data indicating poses of the trackers affixed to the objects according to at least 5 DOF in the known coordinate system. Specifically, the localizer camera 20 may be communicatively coupled to the navigation controller 24 of the surgical navigation system 12 and may be configured to generate and communicate the image data to the navigation controller 24 that indicates the poses of the trackers according to at least 5 DOF in the known coordinate system. The navigation controller 24 may also receive motion measurement data from at least one of the trackers that indicates an angle of inclination of the tracker, or more particularly of a motion sensor of the tracker, relative to gravity. As described in more detail below, the navigation controller 24 may be configured to generate tracker pose data indicative of the poses of the trackers in the known coordinate system according to 6 DOF based on the image data and the motion measurement data, and thereafter determine object pose data indicative of the poses of the objects in the known coordinate system according to 6 DOF based on the tracker pose data and predetermined positional relationships between the objects and trackers.

The localizer camera 20 may have an outer casing 36 that houses at least two optical sensors 38. Each of the optical sensors 38 may be adapted to detect light signals of a particular frequency band that are transmitted by the trackers, such as nonvisible light signals (e.g., infrared, or ultraviolet). While FIG. 1 illustrates the localizer camera 20 as a single unit with multiple optical sensors 38, in an alternative example, the localizer camera 20 may include separate units arranged around the surgical workspace, each with a separate outer casing 36 and one or more optical sensors 38.

The optical sensors 38 may be one-dimensional or two-dimensional charge-coupled devices (CCDs). For example, the outer casing 36 may house two two-dimensional CCDs or three one-dimensional CCDs for triangulating the positions of the optical markers of the trackers in the surgical workspace. Additionally, or alternatively, the localizer camera 20 may employ other optical sensing technologies, such as complementary metal-oxide semiconductor (CMOS) active pixels.

The localizer camera 20 may be mounted to an adjustable arm to selectively position the optical sensors 38 with a field of view of the surgical workspace and target volume that, ideally, is free from obstacles. The localizer camera 20 may be adjustable in at least one degree of freedom by rotating about a rotational joint and may be adjustable about two or more degrees of freedom.

In general, the object to which each tracker is affixed may be rigid and inflexible so that movement of the object cannot or is unlikely to alter the positional relationship between the object and the tracker. In other words, the relationship between a tracker in the surgical workspace and an object to which the tracker is attached may remain fixed, notwithstanding changes in the position of the object within the surgical workspace. For instance, the trackers may be firmly affixed to patient bones and surgical instruments, such as retractors and the surgical instrument 16. In this way, responsive to determining a position of a tracker in the surgical workspace using the localizer camera 20 and/or motion measurement data received from tracker, the navigation controller 24 may infer the position of the object to which the tracker is affixed based on the determined position of the tracker.

For example, when the target volume to be treated is located at the patient's P spine, a tracker 40 may be firmly affixed to each of one or more vertebra V of the patient P, and a tracker 42 may be firmly affixed to the surgical instrument 16. The tracker 42 may be integrated into the surgical instrument 16 during manufacture or may be separately mounted to the surgical instrument 16 in preparation for a surgical procedure. Each vertebra tracker 40 may be directly mountable to a distinct vertebra V, and/or may be removably coupleable to pedicle screws already inserted into the vertebra V.

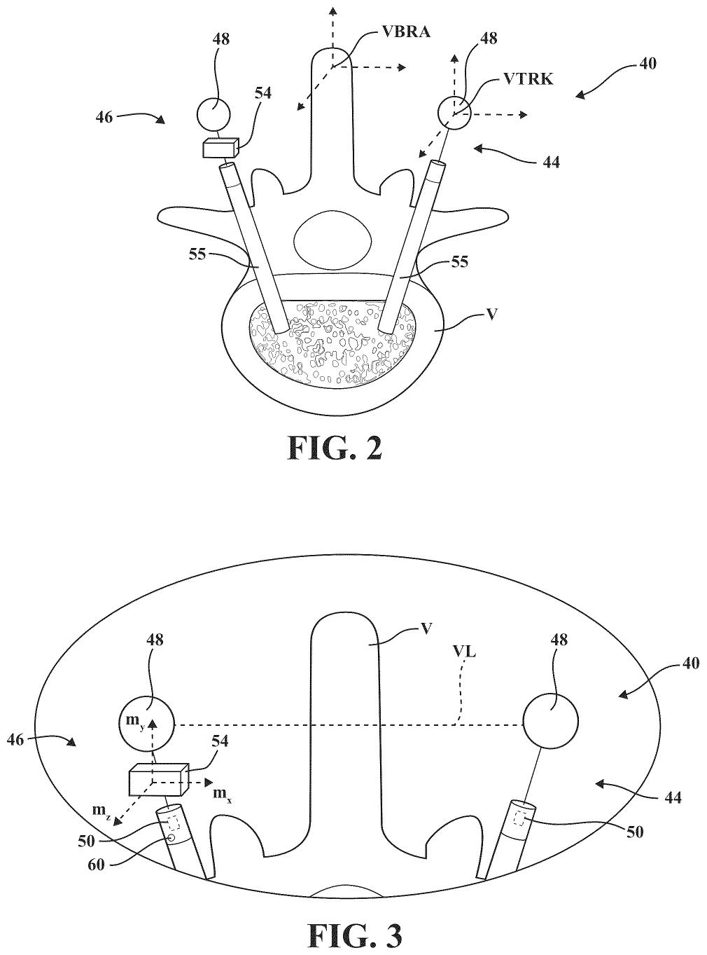

FIGS. 2 and 3 illustrate an example implementation of a vertebra tracker 40 that may be used to track individual vertebra V during a spinal procedure such as illustrated in FIG. 1. As shown in the illustrated example, the vertebra tracker 40 may include first and second tracker assemblies 44, 46, each configured to be rigidly to the vertebra V at a different location. The tracker assemblies 44, 46 may each include a single marker 48 that direct light signals to the optical sensors 38 of the localizer camera 20. In some implementations, the markers 48 may be realized as active markers that receive an electrical current from a power source to generate and emit light signals to the optical sensors 38. In this case, the tracker assemblies 44, 46 may each be powered by an internal battery, or may have leads to receive power through the navigation controller 24. For instance, the markers 48 may be light emitting diodes (LEDs) that transmit light, such as nonvisible light (e.g., infrared, or ultraviolet light), towards the optical sensors 38.

Each tracker assembly 44, 46 may also include a tracker controller 50 communicatively coupled to the markers 48 and to the navigation controller 24. The tracker controllers 50 may be configured to control the rate and order in which the markers 48 fire, such as at the direction of the navigation controller 24. For example, the tracker controllers 50 may cause the marker 48 of each assembly 44, 46 to fire at different rates and/or times to facilitate differentiation of the markers 48 by the navigation controller 24. In some examples, the navigation controller 24 may form a bi-directional infrared communication channel with each tracker controller 50 to control the timing of the firing of the active marker 48 operated by the tracker controller 50, write/read nonvolatile data, and get the status (e.g., battery level, broken LEDs) of the tracker assembly 44, 46 or the object to which the tracker assembly 44, 46 is affixed.

Rather than being active markers, markers 48 of the tracker assemblies 44, 46 may be realized as passive markers, such as reflectors that reflect light emitted from the localizer camera 20. To this end, the localizer camera 20 may include a light source 52 (FIG. 1) that illuminates the tracker assemblies 44, 46 with light, such as nonvisible light (e.g., infrared, or ultraviolet). The markers 48 may be configured to reflect the light back towards the localizer camera 20, which may then be detected by the optical sensors 38.

In some instances, the surgical workspace, or more particularly the tracker 40, may include a combination of active and passive markers 48 for tracking various objects in the surgical workspace. For instance, the marker 48 of the tracker assembly 44 may be realized as a passive marker, in which case the tracker assembly 44 may omit the tracker controller 50. Conversely, the marker 48 of the tracker assembly 46 may be realized as an active marker.

The two markers 48 may enable the navigation controller 24 to determine a pose of the tracker 40 in 5 DOF. More specifically, the two markers 48 may enable the navigation controller 24 to determine a pose of the tracker 40 according to three positional degrees of freedom and two rotational degrees of freedom, but not a rotational degree of freedom relative to a virtual line VL extending between the markers 48 of the tracker assemblies 44, 46. To enable tracking the tracker 40, and correspondingly the vertebra V, in 6 DOF, and thereby provide a full understanding of the pose of the vertebra V, at least one of the tracker assemblies 44, 46 (e.g., the tracker assembly 46) may also include a motion sensor 54. The motion sensor 54 may generally be configured to generate measurement data indicative of an angle of inclination of the tracker 40 that corresponds to the remaining rotational degree of freedom, which may be transmitted to the navigation controller 24 via the tracker controller 50 of the tracker assembly 46.

The motion sensor 54 may be configured to measure movement of the motion sensor 54 relative to an axis m_{z}, or more particularly an angle of inclination of an axis m/with respect to gravity, with the axis m_{z} of the motion sensor 54 being oriented relative to the virtual line VL extending between the optical markers 48 of the first and second tracker assemblies 44, 46 such that the angle of inclination of the axis m_{z} corresponds to rotation of the tracker 40 around the virtual line VL. The navigation controller 24 may thus be configured to utilize measurements from the motion sensor 54 to obtain an angle of inclination of the axis m_{z} with respect to gravity, such as an angle between the axis m_{z} and a plane normal to the gravity vector, and determine an orientation of the tracker 40 in the known coordinate system according to a rotational degree of freedom defined about the virtual line VL based on the angle of inclination.

In some implementations, the tracker assemblies 44, 46 may be secured to the vertebra V such that axis m_{z} is substantially perpendicular to the virtual line VL (e.g., such that the navigation controller 24 is able to track the tracker 40 with sufficient accuracy for the given application, for example within 3 mm, by assuming the axis m_{z} is perpendicular to the virtual line VL). Additionally, or alternatively, such as when the axis m_{z} is not substantially perpendicular to the virtual line VL, prior to initiating tracking of the vertebra V during a surgical procedure, the navigation controller 24 may be configured to determine compensation data indicative of an angle between the axis m_{z} and a plane normal to the virtual line VL. More specifically, the navigation controller 24 may be configured to determine an angle between the axis m_{z} and a projection of the axis m_{z} on the plane. Thereafter, when tracking the vertebra V, the navigation controller 24 may be configured to determine the orientation of tracker 40 according to the further rotational degree of freedom defined about the virtual line VL based on the angle of inclination with respect to gravity obtained during the procedure and the compensation data.

In some implementations, the motion sensor 54 may be realized as an accelerometer, or more particularly a 3-axis accelerometer, configured to enable determination of the remaining rotational degree of freedom not provided by the two optical markers 48 by indicating an inclination of an axis m_{z} of the accelerometer with respect to gravity, which may be disposed to correlate to rotation about the remaining degree of freedom. Correspondingly, the tracker 40 may be tracked in 6 DOF without incorporating a gyroscope, magnetometer, or redundant markers and/or sensors in cooperation with a Kalman-like filter, the implementation of which would require more space and computation and could be subject to unbounded drift and magnetic disturbances in the operating room, respectively. In this way, the tracker 40 may have the benefit of minimal setup and footprint, low computational requirements, and less drift relative to alternative solutions.

Given the rigid nature of the vertebra V, the tracker assemblies 44, 46 once coupled thereto have a fixed positional relationship relative to each other, and thus define a tracker coordinate system VTRK including features of the vertebra tracker 40, such as the markers 48 and motion sensor 54, located at fixed coordinates. The tracker coordinate system VTRK may also have a fixed relationship relative to a vertebra coordinate system VBRA of the vertebra V. Correspondingly, responsive to determining a pose of the vertebra coordinate system VTRK according to 6 DOF relative to a known coordinate system, the pose of the vertebra coordinate system VBRA, and correspondingly of the vertebra V, according to 6 DOF relative to the known coordinate system may be inferred based on the fixed positional relationship between the vertebra coordinate system VBRA and the tracker coordinate system VTRK. In some implementations, the tracker assemblies 44, 46 may be removably and rigidly coupleable to respective pedicle screws 55 inserted into the vertebra V during the course of the procedure to provide such fixed relationships.

By realizing the vertebra tracker 40 as two separate tracker assemblies 44, 46 each including a single marker 48 and one including a motion sensor 54, the footprint of the tracker 40 may be reduced relative to providing a single tracker assembly including three or even two optical markers. Consequently, the surgical workspace is less crowded, simplifying manipulation of the surgical instrument 16 relative to the surgical site and thus making the minimally invasive surgery increasingly suitable for surgical navigation.

In addition or alternative to an accelerometer, the motion sensor 54 may include a gyroscope for measuring the angle inclination of the axis m_{z}, and/or may include a magnetometer for measuring a rotation of the motion sensor 54 relative to gravity. The latter measurement may be used to determine an angle between the axis m_{z} and a plane normal to the virtual line VL, which may be used by the navigation controller 24 to guide a user in placing the tracker 40 such that the axis m_{z} of the motion sensor 54 is substantially perpendicular to the virtual line VL, or to determine the above-described compensation data.

It is further contemplated that the tracker 40 may incorporate one or more redundant markers and/or motion sensors, the latter of which may include any one or more of an accelerometer, gyroscope, or magnetometer, such as to support a Kalman filter implemented by the surgical navigation system 12 and/or to facilitate tracking the tracker 40 when one of the markers 48 is occluded. For example, in some implementations, each of the tracker assemblies 44, 46 may include a motion sensor 54 for measuring motion of the tracker assembly 44, 46 relative to one or more axes, or more particular an angle of inclination of the each of the axes relative to gravity. In some implementations, the motion sensor 54 of one of the tracker assemblies 44, 46 may be generally powered off during tracking, and may be powered on by the navigation controller 24 when needed, such upon detection of an occlusion of the corresponding optical marker 48. It will be appreciated that in an implementation where only one of the tracker assemblies 44, 46 includes an active motion sensor 54, upon occlusion of one of the optical markers 48, the navigation controller 24 may still be able to track the tracker 40 in at least five DOF (e.g., by determining three positional DOF based on the tracked position of the nonoccluded marker 48, and two rotational DOF based on angle of inclination measurements from the motion sensor 54).

It is also contemplated that the two markers 48 and motion sensor 54 may be part of a same tracker assembly configured to be mounted to the patient anatomy such as bone. In other words, each of these components may be supported by a same carrier structure and may have a known relationship relative to the others prior to be mounted to the patient anatomy. As an example, at least two optical markers 48, such as LEDs, may be placed along a single pin structure in line with a motion sensor 54 also incorporated in the pin structure, such that the line of optical markers 48 is substantially perpendicular with an axis of the motion sensor 54 as described herein. In some implementations, rather than LEDs, the optical markers 48 may be formed from an optical fiber extending along the pin structure. The optical fiber may be coupled to a light source such as a laser and have transparent portions/apertures separated along its length to emit light and thus function as the optical markers 48. In yet further implementations, the optical fiber may include a continuous transparent portion extending along its length so as to emit light in the form of a continuous line running along the pin structure, such that the illuminated line is substantially perpendicular with an axis of a motion sensor 54 incorporated in the pin like structure.

Referring again to FIG. 1, prior to the start of a surgical procedure using the surgical system 10, pre-operative images may be generated for anatomy of interest, such as anatomical structures defining and/or adjacent a target volume of patient tissue to be treated by the surgical instrument 16. For example, when the target volume of patient tissue to be treated is in the patient's spine area, pre-operative images of the patient's spinal column may be taken. These images may be based on MRI scans, radiological scans, or computed tomography (CT) scans of the patient's anatomy, and may be used to develop virtual models of the anatomical structures.

Each virtual model for an anatomical structure may include a three-dimensional model (e.g., point cloud, mesh, CAD) includes data representing the entire or at least a portion of the anatomical structure, and/or data indicating a portion of the anatomical structure to be treated, relative to a three-dimensional coordinate system of the anatomical structure. These virtual models may be provided to and stored in the navigation controller 24 in advance of a surgical procedure. In addition, or alternatively to taking pre-operative images, plans for treatment can be developed in the operating room from kinematic studies, bone tracing, and other methods. These same methods may also be used to generate the virtual models described above.

In addition to virtual models corresponding to the patient's anatomical structures of interest, prior to the surgical procedure, the navigation controller 24 may receive and store virtual models for other tracked objects of interest, such as surgical instruments and other objects potentially present in the surgical workspace (e.g., the surgeon's hand and/or fingers). The navigation controller 24 may also receive and store a virtual model for each tracker disposed in the surgical workspace, and relationship data for each tracker indicating a positional relationship between the tracker and the object to which the tracker is affixed. Such positional relationship may be defined relative to the virtual models of the tracker and object. For instance, relative to a given tracker 40 affixed to a vertebra V, the navigation controller 24 may receive data indicating a pose of the vertebra coordinate system VBRA relative to the tracker coordinate system VTRK. In this way, responsive to identifying the pose of the tracker coordinate system VTRK in 6 DOF relative to a known coordinate system, the navigation controller 24 may reference the relationship data for the tracker 40 to determine the pose of the vertebra coordinate system VBRA, and correspondingly of the vertebra V, relative to the known coordinate system.

In some examples, the positional relationship between each tracker and the object to which the tracker is affixed may be indicated manually via interaction with a patient image depicting the tracker on the user interface 28. Alternatively, the positional relationship between each tracker and the object to which the tracker is affixed may be determined by tracing the object with a pointer instrument 56 having its own fixed tracker 58 that is tracked by the surgical navigation system 12 during the tracing, with the surgical navigation system 12 also concurrently tracking the tracker affixed to the object to correlate a pose of the traced object to a pose of the affixed tracker.

The navigation controller 24 may also receive and store surgical plan data prior to a procedure. The surgical plan data may identify the patient anatomical structures involved in the surgical procedure, may identify the instruments being used in the surgical procedure, and may define the planned trajectories of instruments and the planned movements of patient tissue during the surgical procedure.

Prior to continuously tracking a tracker 40 affixed to a vertebra V, the navigation controller 24 may be configured to implement an initialization phase to facilitate determining a virtual model for the tracker 40. To this end, the tracker 40 may include an interface 60 for temporarily coupling at least one further optical marker 48 after the tracker assemblies 44, 46 are secured to the vertebra V. Alternatively, the navigation controller 24 may prompt the user to touch off the interface 60 with the pointer instrument 56. In either case, the navigation controller 24 may be configured to determine a position of the interface 60 with respect to the optical markers 48 of the tracker assemblies 44, 46 based on the emitted light signals.

The motion sensor 54, and more particularly the axis mx of the motion sensor 54, may have a known positional relationship relative to the interface 60 and at least one of the optical markers 48 of the tracker assemblies 44, 46. The navigation controller 24 may thus detect the positions of the optical markers 48 of the tracker assemblies 44, 46 and interface 60 in the known coordinate system as described herein, and may use such detection positions to define the tracker coordinate system VTRK of the vertebra tracker 40, which in turn may define the virtual model for the tracker 40. The detected positions of the optical markers 48 of the tracker assemblies 44, 46 and interface 60 may also define an initial pose of the coordinate system VTRK of the vertebra tracker 40 relative to a known coordinate system, which may be stored by the navigation controller 24 for tracking the pose of the tracker 40 during a tracking phase, along with an initial angle of inclination of the axis m_{z} with respect to gravity that is determined from the motion sensor 54 and corresponds to the initial pose of the tracker coordinate system VTRK, as described in more detail below.

In some implementations, the navigation controller 24 may also be configured to determine the above-described compensation data based on the detected positions of the optical markers 48 of the tracker assemblies 44, 46 and interface 60, such as based on a predefined relationship between the motion sensor 54, the optical marker 48 of the tracker assembly 46, and the interface 60. In alternative implementations, the navigation controller 24, based on the detected positions of the optical markers 48 of the tracker assemblies 44, 46 and interface 60, may be configured to guide the practitioner in orientating the tracker assembly 46 such that the axis m_{z} of the motion sensor 54 is substantially perpendicular to the virtual line VL extending between the optical markers 48.

Referring again to FIG. 1, in some implementations, the localizer camera 20 may further include a motion sensor 62, similar to the motion sensor 54, configured to generate data indicative of an angle inclination of an axis of the localizer camera 20, such as defined by the localizer coordinate system LCLZ, relative to gravity. After securing the tracker 40 to the vertebra V and during the initialization phase, the navigation controller 24 may be configured to similarly utilize measurement data from the motion sensor 62 to determine an initial angle of inclination of the motion sensor 62 with respect to gravity, and to determine an initial pose of the tracker coordinate system VTRK in the known coordinate system according to 6 DOF based on the initial angle of inclination of the motion sensor 54, the initial angle of inclination of the motion sensor 62, and positions of the optical markers 48 of the tracker assemblies 44, 46 detected by the navigation controller 24 as described herein. More specifically, the navigation controller 24 may be configured to determine an initial pose of the tracker coordinate system VTRK in the known coordinate system according to three positional and two rotational DOF based on the tracked positions of the optical markers 48, and determine an initial pose of the tracker coordinate system VTRK in the known coordinate system according to the further rotational DOF based on the initial angle of inclination of the motion sensor 54, the initial angle of inclination of the motion sensor 62, and a predetermined relationship between the virtual line VL extending between the optical markers 48 and the axis m_{z} (e.g., calibration data described above). Utilizing the motion sensor 62 to determine the initial pose of the tracker coordinate system VTRK may be as a redundancy or in place of using the interface 60 as described above.

Moreover, during tracking of the tracker 40 during the tracking phase, the navigation controller 24 may be configured to determine the orientation of the tracker coordinate system VTRK in the known coordinate system according to the rotational degree of freedom defined about the virtual line VL based on an updated angle of inclination obtained from the motion sensor 54, the initial angle of inclination determined from the motion sensor 54, and the initial angle of inclination determined from the motion sensor 62. As an example, the navigation controller 24 may be configured to utilize the motion sensor 62 as a bump detector, such as by determining whether the measurement data from the motion sensor 62 indicates a change in the pose of the localizer camera 20, and correspondingly the localizer coordinate system LCLZ. If so, then the navigation controller 24 may be configured to reestablish an initial pose of the tracker coordinate system VTRK as described above. The navigation controller 24 may be configured to indicate when such process is occurring via the user interface 28. As will be described in the subsequent section, the motion sensor 62 can be employed in techniques to determine whether any of the first and second tracker assemblies 44, 46 has been bump, moved, dislocated, or dislodged relative to the bone.

As noted above, the surgical instrument 16 may form part of an end effector 18 of the robotic manipulator 14. The robotic manipulator 14 may include a base 64, several links 66 extending from the base 64, and several active joints 68 for moving the surgical instrument 16 with respect to the base 64. The links 66 may form a serial arm structure as shown in FIG. 1, a parallel arm structure, or other suitable structure. The robotic manipulator 14 may include an ability to operate in a manual mode in which a user grasps the end effector of the robotic manipulator 14 to cause movement of the surgical instrument 16 (e.g., directly, or through force/torque sensor measurements that cause active driving of the robotic manipulator 14). The robotic manipulator 14 may also include a semi-autonomous mode in which the surgical instrument 16 is moved by the robotic manipulator 14 along a predefined tool path (e.g., the active joints 68 of the robotic manipulator 14 are operated to move the surgical instrument 16 without requiring force/torque on the end effector from the user). An example of operation in a semi-autonomous mode is described in U.S. Pat. No. 9,119,655 to Bowling, et al., which is hereby incorporated by reference in its entirety. A separate tracker 42 may be attached to the base 64 of the robotic manipulator 14 to also track movement of the base 64 by the localizer camera 20.

Similar to the surgical navigation system 12, the robotic manipulator 14 may house a manipulator controller 70 configured to implement the functions, features, and processes of the robotic manipulator 14 described herein. The surgical instrument 16 may be a powered surgical instrument, and may similarly include a localizer controller 72 configured to implement the functions, features, and processes of the surgical instrument 16, including controlling actuation of the of the end effector 18 to treat target tissue, such as at the direction of the manipulator controller 70 and/or navigation controller 24 based on the pose or position of the end effector 18 relative to the target tissue.

During a surgical procedure, the manipulator controller 70 may be configured to determine a desired location to which the surgical instrument 16 should be moved, such as based on navigation data received from the navigation controller 24. Based on this determination, and information relating to the current position of the surgical instrument 16, the manipulator controller 70 may be configured to determine an extent to which the links 66 need to be moved to reposition the surgical instrument 16 from the current position to the desired position. Data indicating where the links 66 are to be repositioned may be forwarded to joint motor controllers (e.g., one for controlling each motor) that control the active joints 68 of the robotic manipulator 14. Responsive to receiving such data, the joint motor controllers may be configured to move the links 66 in accordance with the data, and consequently move the surgical instrument 16 to the desired position.

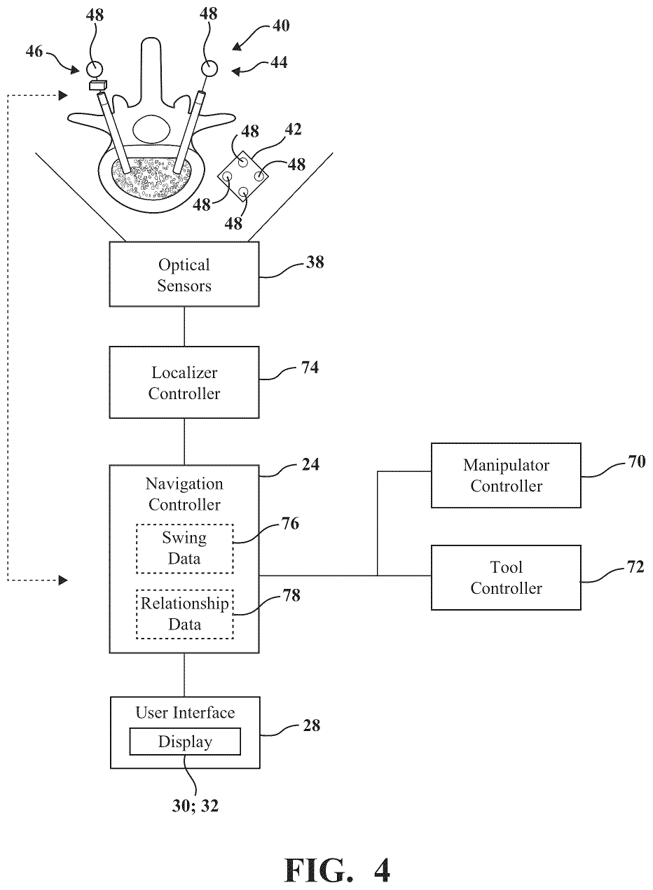

Referring now to FIG. 4, the localizer camera 20 may include a localizer controller 74 communicatively coupled to the optical sensors 38 and to the navigation controller 24. The tracker 42 may include at least three optical markers 48 arranged according to a predefined arrangement, and assuming the optical markers 48 are active, may also include a controller in communication with the navigation controller 24 to regulate the firing of the optical markers 48. During a surgical procedure, the localizer controller 72 may be configured to operate the optical sensors 38 to cause them to generate optical-based signals indicative of detected light signals received from the trackers 40, 42, or more particularly indicative of the image plane positions of the optical sensors 38 in which such light signals were detected. The sampling rate of the optical sensors 38 is the rate at which the optical sensors 38 detect light signals from sequentially fired markers 48. The optical sensors 38 may have sampling rates of 100 Hz or more, or more preferably 300 Hz or more, or most preferably 500 Hz or more. In one instance, the optical sensors 38 may have sampling rates of 8000 Hz.

Responsive to the optical sensors 38 receiving light signals from the trackers 40, 42, the optical sensors 38 may output optical-based signals to the localizer controller 74 indicating the positions of the markers 48 of the trackers 40, 42 relative to the localizer camera 20, which may be used by the navigation controller 24 to determine poses of the objects affixed to the trackers 40, 42 relative to the localizer camera 20. In particular, each optical sensor 38 may include a one- or two-dimensional sensor area (also referred to as an “image plane”) that detects light signals from the trackers 40, 42, and responsively outputs optical-based signals indicating pixel coordinates within the sensor area that each light signal was detected. The optical-based signals output from each optical sensor 38 may thus represent an image of the trackers 40, 42 generated by the optical sensor 38 from the detected light signals, with the image including blobs in pixel coordinates corresponding to the positions in the image plane of the optical sensor 38 that light signals were detected. The detected position of each light signal may be based on the angle at which the light signal is received by the optical sensor 38 and may thus correspond to the position of the marker 48 in the surgical workspace that emitted the detected light signal towards the optical sensor 38.

The optical sensors 38 may communicate the optical-based signals to the localizer controller 74, which in turn may generate image data for each optical sensor 38 based on the optical-based signals received from the optical sensor 38 and communicate such image data to the navigation controller 24. The image data for an optical sensor 38 may indicate the image and/or image plane positions represented by the optical-based signals received from the optical sensor 38. Contemporaneously with operation of the optical sensors 38 to image the trackers 40, 42, the navigation controller 24 may communication with the motion sensor 54 of each tracker 40, such as via the tracker controller 50 of the tracker assembly 46, to receive motion measurement data that may indicate an angle of inclination of the motion sensor 54 relative to gravity.

The navigation controller 24 may then generate tracker pose data indicating the poses of the trackers 40, 42 relative to the localizer camera 20 based on the received image data and motion measurement data. More particularly, the navigation controller 24 may determine a position of the markers 48 in the localizer coordinate system LCLZ based on the image data. For instance, the navigation controller 24 may be configured to correlate blobs corresponding to a same optical marker 48 in the image data, triangulate the positions of the optical markers 48 relative to the localizer camera 20 based on the positions of the correlated blobs in the image data and a known positional relationship between the optical sensors 38, and assign the triangulated positions to the markers 48 of each tracker 40, 42 based on a known geometry of the markers 48 of each tracker 40, 42, or alternatively based on a known frequency, timing, or intensity associated with each marker 48.

The navigation controller 24 may then be configured to generate tracker pose data indicating the pose of each tracker 40, 42 relative to the localizer coordinate system LCLZ according to 6 DOF. Relative to the trackers 42 each including at least three markers 48, the determined positions of the markers 48 of each tracker 42 relative to the localizer coordinate system LCLZ may define the pose of tracker 42 according to 6 DOF. Conversely, relative to the vertebra trackers 40 having two markers 48, the determined positions of the markers 48 of each tracker 40 relative to the localizer coordinate system LCLZ may define the pose of tracker 40 according to 5 DOF. The navigation controller 24 may be configured to fuse 5 DOF pose of each tracker 40 with the motion measurement data received from the tracker 40 to determine the pose of the tracker 40 according to 6 DOF.

In some implementations, the navigation controller 24 may be configured to access previously stored swing data 76 based on the motion measurement data, which may indicate an orientation of the tracker 40 according to the rotational degree freedom defined about the virtual line VL relative to the angle of inclination indicated by the motion measurement data. More particularly, following production of the tracker 40 but prior to distribution, the tracker 40 may be disposed in a swing table configured to position the tracker 40 in various orientations with respect to gravity. For each orientation, a position of the optical markers 48 and/or interface 60 of the tracker 40 may be obtained using a coordinate measurement machine (CMM) and be stored in association with an angle of inclination indicated by the motion sensor 54. Such data may be used to define the positional relationship of the motion sensor 54 relative to the one or more of the optical markers 48 and/or the interface 60, and/or to develop a formula for estimating an orientation of the tracker coordinate system VTRK relative to the angle of inclination indicated by the motion sensor 54, each of which may be indicated by the swing data 76.

Based on the determination of the pose of the tracker 40, 42 according to 6 DOF in the known coordinate system, the navigation controller 24 may be configured to generate object pose data indicating the poses of the objects affixed to the trackers 40, 42 relative to the localizer camera 20 according to 6 DOF based on the tracker pose data. Specifically, the navigation controller 24 may retrieve previously stored relationship data 78 indicating the relationships between the trackers 40, 42 and the objects to which the trackers 40, 42 are affixed, including the vertebra V, and may apply these positional relationships to the tracker pose data to determine the poses of the objects fixed to the trackers 40, 42 relative to the localizer camera 20 according to 6 DOF.

In alternative implementations, the localizer controller 74 may be configured to determine the tracker pose data and/or object pose data based on the optical-based signals generated by the optical sensors 38, and to transmit the tracker pose data and/or object pose data to the navigation controller 24 for further processing.

Each of the controllers described herein may include a processor, memory, and non-volatile storage each operatively coupled to the processor. The processor may be programmed to perform the functions, features, and processes of the controller described herein, and may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions stored in the memory. The memory may include a single memory device or a plurality of memory devices including, but not limited to, read-only memory (ROM), random access memory (RAM), volatile memory, non-volatile memory, static random access memory (SRAM), dynamic random access memory (DRAM), flash memory, cache memory, or any other device capable of storing information. The non-volatile storage may include one or more persistent data storage devices such as a hard drive, optical drive, tape drive, non-volatile solid-state device, or any other device capable of persistently storing information.

The non-volatile storage may store software, which may include one or more applications and/or modules embodied by a set of computer-executable instructions compiled or interpreted from a variety of programming languages and/or technologies, including, without limitation, and either alone or in combination, Java, C, C++, C#, Objective C, Fortran, Pascal, Java Script, Python, Perl, and PL/SQL. The processor may operate under control of the software stored in the non-volatile storage. In particular, the processor may be configured to read into the memory and execute the computer-executable instructions embodying the software. Upon execution by the processor, the computer-executable instructions may be configured to cause the processor to implement the configured functions, features, and processes of the controller described herein.

The non-volatile storage may also store data that facilitates operation of the controller. Specifically, the software of the controller may be configured upon execution to access the data to facilitate implementation of the functions, features, and processes of the navigation controller 24 described herein. For example, relative to the navigation controller 24, the data stored din the non-volatile storage may include the swing data 76 and relationship data 78 described herein.

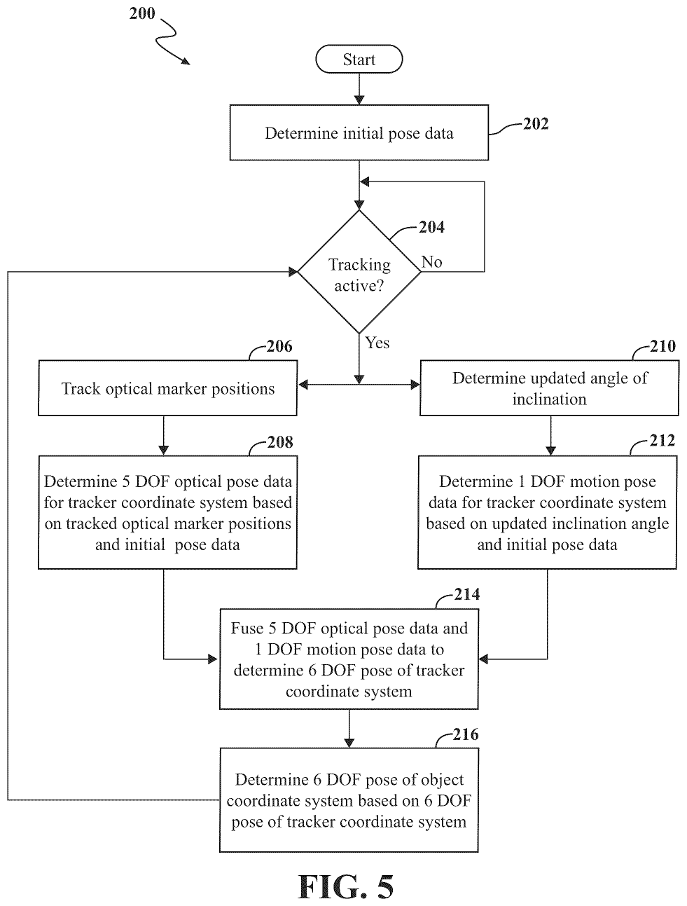

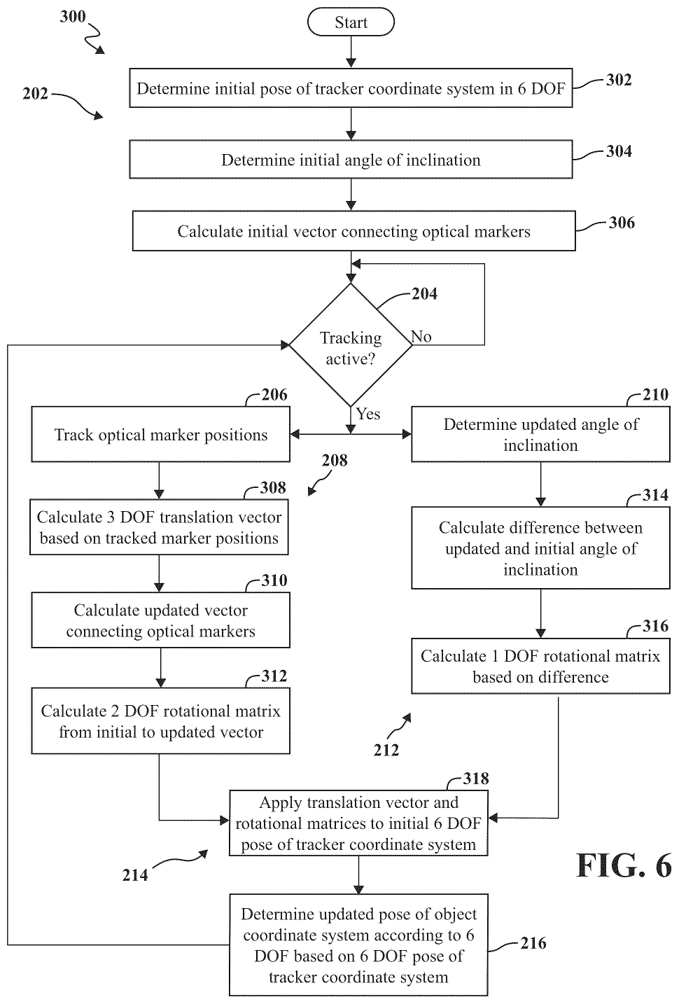

FIG. 5 illustrates a method 200 for determining a pose of a vertebra V in 6 DOF using the tracker 40, and FIG. 6 illustrates a method 300 setting forth an exemplary implementation of the method 200. The methods 200, 300 may each be implemented by one or more of the controllers described herein, such as the navigation controller 24.

In block 202, the method 200 may include determining initial pose data of the tracker 40. In some implementations, determining the initial pose data may include one or more of blocks 302, 304, and 306 of the method 300. In block 302, the method 300 may include determining an initial pose of the tracker coordinate system VTRK in a known coordinate system, such as the localizer coordinate system LCLZ, according to 6 DOF. As described above, such initial pose may be determined by imaging the tracker 40 secured to the vertebra V with a localizer camera 20 when an additional optical marker 48 is temporarily disposed relative to the other markers 48 of the tracker 40, such via an interface 60 of the tracker 40. In block 304, the method 300 may include determining an initial angle of inclination relative to gravity corresponding to the initial pose, such as utilizing measurements from the motion sensor 54. More specifically, an angle of inclination of an axis m_{z} of the motion sensor 54 relative to gravity may be determined, the inclination angle of the axis having a known relationship to the rotation of the tracker coordinate system VTRK around a virtual line VL connecting the markers 48 of the tracker 40. In block 306, the method 300 may include calculating an initial vector connecting the optical markers 48 of the first and second tracker assemblies 44, 46 based on the initial pose of the tracker coordinate system VTRK determined above.

In block 204, the methods 200, 300 may each include determining whether to activate tracking of the tracker 40. For instance, a user may interact with the user interface 28 of the surgical navigation system 12 to indicate the start of the surgical procedure and/or to begin a tracking phase of the tracker 40. Responsively, it may be determined to activate tracking of the tracker 40.

Responsive to determining to activate tracking (“Yes” branch of block 204), in block 206, the methods 200, 300 may each include tracking positions of the optical markers 48 of the first and second tracker assemblies 44, 46 relative to a known coordinate system, such as the localizer coordinate system LCLZ and using the localizer camera 20. In block 208, the method 200 may include determining 5 DOF optical pose data indicating an updating pose for the tracker coordinate system VTRK associated with the first and second tracker assemblies 44, 46 in the known coordinate system according to three positional degrees of freedom and two rotational degrees of freedom based on the tracked positions of the optical markers 48 and the initial pose data.

Block 208 of the method 200 may include one or more of blocks 308, 310 and 312 of the method 300. In block 308, the method 300 may include calculating a 3 DOF translation vector from the initial positions of the markers 48 of the tracker 40 as indicated by the initial pose to the tracked positions of the markers 48 as determined in block 206 in the known coordinate system. In block 310, the method 300 may include calculating an updated vector connecting the optical markers 48 of the first and second tracker assemblies 44, 46 based on the tracked positions of the optical markers 48. In block 312, the method 300 may include calculating a 2 DOF rotational matrix from the initial vector connecting the optical markers 48 to the updated vector in the known coordinate system.

In block 210, the methods 200, 300 may each include obtaining an updated angle of inclination indicated by the motion sensor 54. As previously described, the angle of inclination may have a known relationship to the rotation of the tracker coordinate system VTRK around the virtual line VL connecting the markers 48 of the tracker 40. More specifically, the difference between the updated angle and the initial of angle of inclination determined in block 304 may indicate a change in orientation of the tracker coordinate system VTRK according to a rotational degree of freedom defined about the virtual line VL relative to an initial orientation of the tracker coordinate system VTRK. In block 212, the method 200 may thus include determining 1 DOF motion pose data indicating a change in orientation of the tracker coordinate system VTRK according to the further rotational degree of freedom defined about the virtual line VL based on the updated angle of inclination and the initial pose data.

More specifically, block 212 may include one or more of blocks 314 and 316 of the method 300. In block 314, the method 300 may include calculating a difference between the updated angle of inclination and the initial angle of inclination. In block 318, the method 300 may include calculating a 1 DOF rotational matrix based on the difference. This calculation may be based on the swing data 76, which as described above may indicate a rotation of the tracker coordinate system VTRK relative to the virtual line VL extending between the markers 48 of the tracker 40.

In block 214, the method 200 may include fusing the 5 DOF optical pose data with the 1 DOF motion pose data to determine a pose of the tracker coordinate system VTRK in 6 DOF. More specifically, block 214 may include determining a pose of a tracker coordinate system VTRK associated with the tracker assemblies 44, 46 in the localizer coordinate system according to 3 positional degrees of freedom and two rotational degrees of freedom based on the tracked positions of the optical markers, such as by applying the 5 DOF optical pose data to the initial pose of the tracker coordinate system VTRK, and determining an orientation of the tracker coordinate system VTRK according to a further rotational DOF defined about the virtual line VL extending between the optical markers 48 of the tracker assemblies 44, 46 based on the obtained angle of inclination, such as by application of the 1 DOF motion pose data to the initial pose of the tracker coordinate system VTRK.

Block 214 may include block 318 of the method 300. In block 318, the method 300 may include translating the tracker coordinate system VTRK from the initial pose using the 3 DOF translation vector, and rotating the tracker coordinate system VTRK from the initial pose based on the 2 DOF rotational matrix. Correspondingly, the pose of the tracker coordinate system VTRK in the known coordinate system according to three positional DOF and two rotational DOF may be determined. Block 318 may then include rotating the tracker coordinate system VTRK from the initial pose using the 1 DOF rotational matrix. Correspondingly, an orientation of the tracker coordinate system VTRK according to a further rotational degree of freedom defined about the virtual line VL extending between the optical markers 48 of the tracker assemblies 44, 46 may also be determined, resulting in a pose of the tracker coordinate system VTRK according to 6 DOF in the known coordinate system.

In block 216, the methods 200, 300 may each include determining the pose of the vertebra V in the known according to 6 DOF based on the 6 DOF pose of the tracker coordinate system VTRK in the known coordinate system and the relationship data 78 indicative of the pose of the vertebra coordinate system VBRA relative to the tracker coordinate system VTRK.

Following block 216, the methods 200, 300 may each return to block 204 to determine whether to continue tracking the tracker 40. For instance, a user may interact with the user interface 28 to indicate the end of the surgical procedure, in which case it may be determined to discontinue tracking. Conversely, responsive to determining to continue tracking (“Yes” branch of block 204), methods 200, 300 may each continue tracking as described above.

Although illustrated as occurring in parallel, it will be appreciated that the block 206 branch of the methods 200, 300 and the block 210 branch of the methods 200, 300 may occur in sequence and/or in a difference order. For example, blocks 206 and 210 may be performed, and thereafter blocks 308, 310, and 312 may be performed, followed by performance of blocks 314 and 316. Similarly, in block 318, the initial pose of the tracker coordinate system VTRK may be transformed in a different order than described above.

### II. Using Motion Sensor(s) for Tracker Accuracy Monitoring

Herein we describe systems, methods, and solutions wherein the one or more controllers utilize one or more motion sensor(s) 62 to monitor accuracy of one or more tracker assemblies 44, 46. Namely, the motion sensor(s) 62 can be used to monitor whether one or more tracker assemblies 44, 46 has been dislocated, dislodged, bumped, or moved relative to the bone. The motion sensor(s) 62 can also detect whether a component or portion of one tracker assembly has been dislocated, dislodged, bumped, or moved relative to other components or portions of the tracker assembly, which is also indicative of an undesirable movement relative to the bone.

By monitoring these undesirable situations, the techniques described herein can better ensure tracking accuracy of the bone by the respective tracker assembly. Moreover, by providing angle of inclination measurements (respect to Earth's gravity), the motion sensor(s) 62 is/are capable of establishing accuracy detection monitoring in a manner that does not require an additional optical marker/tracker for monitoring, and therefore, is not susceptible to optical tracking errors. Additionally, in some solutions, the motion sensor(s) 62 can be seamlessly integrated into or coupled to a single tracker assembly and without necessarily requiring an additional fixation point on the bone separate from the tracker assembly being monitored. Monitoring by the motion sensor(s) 62 is continuous, and therefore, can reduce or eliminate the need for the surgeon to perform a separate verification step to confirm the tracker accuracy by using the navigated pointer 56 to touch off a separately checkpoint that is implanted onto the tracked bone. Accordingly, the described solutions provide continuous and robust tracker accuracy monitoring in a manner that is less invasive to the patient, less susceptible to additional optical tracking errors, seamlessly integrated with tracker configurations, and that reduces surgical procedure time.

These techniques can be utilized in conjunction with the above-described solutions, or entirely separate therefrom. The described implementations can be used in conjunction with the surgical navigation system 12 described above. Hence, all the features, capabilities, components of the surgical navigation system 12 are incorporated by reference in this section and not repeated for simplicity. Any one or more controllers described above, such as the navigation controller 24 and the localizer controller 74 can be employed in the solutions described herein.

The solutions described in this section are not intended to be limited by the implementations above. As will be illustrated in the following description, the motion sensor(s) 62 described in this section may be configured and located differently than the solutions described in the previous section (I). For example, the motion sensor(s) 62 can be located anywhere on a tracker assembly 44, 46 or on the bone. Moreover, the tracker assembly or assemblies 44, 46 may have different configurations from those described in the section above. Any of the tracker assemblies 44, 46 can include any suitable optical tracking configuration, such as any number of optical markers 48, (e.g., one, two, three, four, etc.). In some instances, the any of the tracker assemblies 44, 46 can employ other tracking modalities besides optical tracking, such as radio frequency, electromagnetic, inertial tracking, or the like. Any of the tracker assemblies 44, 46 can be mounted to the bone using any appropriate tracker mounting system. For example, tracker mounting system can include an attachment portion that attaches directly to the bone. The attachment portion can be a bone plate, a bone pin, a bone fastener, a clamp, limb strap, or the like. A stem or post can extend from the attachment portion. The optical marker(s) 48 can be attached to the stem using a tracking head or housing that supports the optical marker(s) 48. The tracking head can be coupled to the stem in a fixed or adjustable manner. In other cases, the optical marker(s) 48 can be supported directly by the stem or extend therefrom (e.g., using posts).

We now will describe two example implementations of this solution. In a first example, at least one motion sensor 62 is used with optical markers 48 to provide a hybrid-inertial tracker accuracy monitoring. In another example, two motion sensors 62 are used, with or without optical marker(s) 48 to provide inertia-inertial tracker accuracy monitoring. In both instances, the motion sensor(s) 62 can detect whether the tracker assembly 44, 46 has been dislocated, dislodged, bumped, or moved relative to the bone or whether a component of one tracker assembly 44, 46 has been dislocated, dislodged, bumped, or moved relative to other components of the tracker assembly 44, 46.

### A. Hybrid Inertial-Optical Tracker Accuracy Monitoring

In one implementation for tracker accuracy monitoring, at least one motion sensor 62 is used with optical markers 48 of the tracker assembly 44, 46. In this example, the tracker assembly 44, 46 is rigidly attached to the bone at a first location. The tracker assembly 44, 46 includes at least one optical marker 48. The motion sensor 62 is rigidly attached to the bone and is configured to measure an angle of inclination with respect to gravity (e.g., gravity vector), as described above. The localizer 20 is configured to optically detect a pose of the at least one optical marker 48. The one or more controllers 24, 74 are coupled to the localizer 20 and the motion sensor 62.

The one or more controllers 24, 74 implement a method described as follows, without limitation to the specific order of steps unless necessitated by their logical order. The one or more controllers 24, 74 receive the measured angle of inclination from the motion sensor 62. The motion sensor 62 can include a communication device (such as Wi-Fi, Bluetooth, RF, infrared, or other near-field communication techniques) to remotely transmit the measured angle of inclination to the one or more controllers 24, 74. The one or more controllers 24, 74 obtain the tracked pose of the at least one optical marker 48. Acquisition of the measured angle of inclination and tracked pose may be done during an initialization phase at the time of tracker setup wherein the tracker assembly 44, 46 remains stationarily and rigidly attached to the bone, as intended, at the first location. The one or more controllers 24, 74 establish a baseline relationship between the at least one optical marker 48 and the motion sensor 62 by combining the detected pose of the at least one optical marker 48 and the measured angle of inclination. The baseline relationship may be a geometric relationship. For example, the coordinates representing the pose of the at least one optical marker 48 and a virtual line representing the measured angle of inclination can be combined in a common coordinate system. In some cases, the pose of the bone, if known, can also be combined with the tracked pose and measured angle of inclination. However, the techniques can operate without relying on the pose of the bone because the motion sensor 62 provides a second reference (aside from the optical marker) that is rigidly attached to the bone.

After completion of this initialization stage, the baseline relationship is established to provide a reference for monitoring relative movement of the tracker assembly 44, 46, component of the tracker assembly 44, 46, or at least one optical marker 48, relative to the bone. The one or more controllers 24, 74 monitor a measured relationship between the detected pose of the at least one optical marker 48 and the measured angle of inclination. That is, the one or more controllers 24, 74 receive over time the detected pose of the at least one optical marker 48 and the measured angle of inclination. These detected pose and measured angle can be received at any appropriate interval and for any duration and can be monitored continuously or discretely. For example, the one or more controllers 24, 74 can obtain the detected pose and measured angle at each camera frame, every second, or every N seconds. This monitoring can occur for any duration after the initialization step and can persist throughout the surgical procedure without interruption.

Through this monitoring, the one or more controllers 24, 74 can detect a deviation between the measured relationship and the baseline relationship to identify that the tracker assembly 44, 46, component of the tracker assembly 44, 46, or at least one optical marker 48, has moved relative to the bone. That is, even if the measured angle of inclination where to change from the baseline, it is the change in the relative relationship between the measured angle and the tracked pose of the optical marker 48 that indicates the error. Upon detecting the deviation, the one or more controllers 24, 74 can be configured to identify an error condition.

In some instances, the one or more controllers 24, 74 can implement a threshold, limit, or detection range to apply to the detected deviation so as to avoid false error alarms or overly sensitive monitoring. For example, the threshold can be a distance error measurement of the at least one optical marker 48 wherein the error is not triggered unless the distance error measurement is greater than the threshold, such as 1 mm or 2 mm. In another example, a threshold error angle can be set between the measured angle of inclination and the at least one optical marker 48 (e.g., 0.5 degrees).

The one or more controllers 24, 74 can be configured to generate feedback to a user in response to detection of the error. In one example, the feedback is provided through the tracker assembly 44, 46. The tracker assembly 44, 46 can include an audible alarm, a visual indicator, or vibratory feedback to alert the user. For instance, the tracker assembly 44, 46 can include a visual indicator, which could be the optical marker 48, or a separate visual indicator. The visual indicator may change color (e.g., green to red) in response to the error. The one or more controllers 24, 74 can command any of this feedback through the navigation system 12 communications to the tracker assembly 44, 46. Additionally or alternatively, the feedback can be provided through the motion sensor 62, e.g., using an audible alarm or through the navigation system 12, e.g., using a visual or audiovisual alert, message or notification provided by the display unit(s) 32, 34.

Having described the solution, several configurations of the motion sensor 62 are contemplated for which the solution can fully be implemented. In one example, the motion sensor 62 is coupled to the tracker assembly 44, 46 itself. In other words, the motion sensor 62 is rigidly attached to the bone by virtue of the tracker assembly 44, 46 to which it is attached. The motion sensor 62 can be integrally coupled to the tracker assembly 44, 46, or detachably coupled (and removable from) the tracker assembly 44, 46. In this implementation, the motion sensor 62 can be coupled to the attachment portion, to the stem, or to any other component of the tracker assembly 44, 46 besides being rigidly fixed to the at least one optical marker 48. This way, the motion sensor 62 is provided at a different component or location than the at least one optical marker 48. In this configuration, the motion sensor 62 can detect whether the component of the tracker assembly (to which it is attached) has moved relative to the at least one optical marker 48, thereby indicating the tracker assembly 44, 46 has experienced a distortion from its original configuration.

In another implementation, the motion sensor 62 is rigidly attached to the bone at a second location spaced apart and different from the first location (at which the “first” tracker assembly 44 is attached). In this configuration, by being spaced apart and separated from the first tracker assembly 44, the motion sensor 62 can detect whether any part of the first tracker assembly 44, including at least one optical marker 48, has moved relative to the bone.

In one example, the motion sensor 62 is coupled to a second tracker assembly (e.g., 46) is rigidly attached to the bone at the second location. The second tracker assembly 46 can include any of the described tracking configurations or modalities (e.g., optical tracking or the like). The motion sensor 62 can be integrally coupled to the second tracker assembly 46, or detachably coupled (and removable from) the second tracker assembly 46. The motion sensor 62 can be coupled to any component of the second tracker assembly 46, including directly adjacent the optical marker(s). In such instances, the tracked pose of the optical marker(s) of the second tracker assembly 46 can optionally be combined to determine the baseline relationship. However, this need not be required as the relationship between the measured angle of inclination can be fixed or moveable relative to the tracked pose of the optical marker(s) of the second tracker assembly 46.

In another example, the motion sensor 62 is embodied in a tracker observation device rigidly attached to the bone at the second location. The tracker observation device may be dedicated device specifically intended for the purpose of remotely monitoring the first tracker assembly 44. The tracker observation device may be used in addition to, or instead of, the second tracker assembly 46. In one configuration, the tracker observation device comprises a body that supports the motion sensor 62. The body of the tracker observation device supports an attachment that is configured to be mounted to the bone at the second location. The attachment can be a bone plate, a bone pin, a bone fastener, a clamp, limb strap, or the like. The body also supports the communication device of the motion sensor 62 to remotely transmit the measured angle of inclination to the one or more controller 24, 74. The motion sensor 62 can be integrally coupled to the tracker observation device 46 or detachably coupled (and removable from) the tracker observation device.

In one example, the tracker observation device can be screwed into the bone at any desired landmark, e.g., like a “checkpoint.” The tracker observation device can have a small footprint so as to minimize interference with the surgical site. For example, the tracker observation device can have a width of 10 mm or less and when installed to the bone, can extend from the surface of the bone only minimally, e.g., 5 mm or less. A distal portion of the tracker observation device may extend from the surface of the bone when the tracker observation device is installed to the bone. This distal portion can include a divot formed therein. The divot can be carefully machined to fit the distal tip of the navigated pointer 56. As such, by virtue of being rigidly attached to the bone, the tracker observation device can optionally and additionally be utilized as a “manual” checkpoint to verify the tracker assembly accuracy. This can be implemented for redundancy checking, e.g., should the surgeon wish to verify operation of the tracker observation device or tracker accuracy.

### B. Inertial-Inertial Tracker Accuracy Monitoring

In another implementation for tracker accuracy monitoring, at least two motion sensors 62 are used one or more tracking assemblies 44, 46. In this example, the tracker assembly 44, 46 is rigidly attached to the bone. The tracker assembly 44, 46 includes at least one optical marker 48. The tracker assembly 44, 46 also includes a (tracker) motion sensor 62 configured to measure a first angle of inclination with respect to gravity, in the manner described above. The tracker motion sensor 62 can be coupled to the tracker assembly 44, 46 in any of the described manners. The second motion sensor, i.e., a tracker observation sensor 62′, is also rigidly attached to the bone. The tracker observation sensor 62′ is configured to measure a second angle of inclination with respect to gravity. The localizer 20 is configured to optically detect a pose of the at least one optical marker 48. The one or more controllers 24, 74 are coupled to the localizer 20, the tracker motion sensor 62, and the tracker observation sensor 62′.

The one or more controllers 24, 74 implement a method described as follows, without limitation to the specific order of steps unless necessitated by their logical order. The one or more controllers 24, 74 receive the measured first and second angles of inclination from the tracker motion sensor 62 and tracker observation sensor 62′. Both sensors 62, 62′ can include the described communication device to remotely transmit their respective measured angle of inclination to the one or more controllers 24, 74. Acquisition of the measured first and second angles of inclination may be done during the described initialization phase at the time of tracker setup wherein the tracker assembly 44, 46 remains stationarily and rigidly attached to the bone, as intended, at the first location. The one or more controllers 24, 74 establish a baseline relationship between the tracker assembly 44, 46 and the tracker observation sensor 62′ by combining the measured first and second angles of inclination. The baseline relationship may be a geometric relationship. For example, the virtual lines representing the measured first and second angles of inclination can be combined in a common coordinate system. Optionally, the one or more controllers 24, 74 can combine the tracked pose of the at least one optical marker 48 with the measured first and second angles of inclination (e.g., in the common coordinate system). Additionally, the pose of the bone, if known, can also be combined with the measured first and second angles of inclination. However, the techniques can operate without relying on the pose of the bone or the at least one optical marker 48 because the two sensors 62, 62′ represent two separate references that are rigidly attached to the bone.

After completion of this initialization stage, the baseline relationship is established to provide a reference for monitoring relative movement of the tracker assembly 44, 46, component of the tracker assembly 44, 46, or at least one optical marker 48, relative to the bone. The one or more controllers 24, 74 track the measured first and second angles of inclination to monitor a measured relationship between the tracker assembly 44, 46 and the tracker observation sensor 62′. That is, the one or more controllers 24, 74 receive over time the measured first and second angles of inclination. These measured first and second angles of inclination can be received at any appropriate interval and for any duration and can be monitored continuously or discretely. This monitoring can occur for any duration after the initialization step and can persist throughout the surgical procedure without interruption.

Through this monitoring, the one or more controllers 24, 74 can detect a deviation between the measured relationship and the baseline relationship to identify that the tracker assembly 44, 46, component of the tracker assembly 44, 46, or at least one optical marker 48, has moved relative to the bone. That is, relative change in relationship between the measured first and second angles of inclination indicates the error. Upon detecting the deviation, the one or more controllers 24, 74 can be configured to identify the error condition.

In some instances, the one or more controllers 24, 74 can implement a threshold, limit, or detection range to apply to the detected deviation so as to avoid false error alarms or overly sensitive monitoring. For example, the threshold can be a distance error measurement of between the measured first and second angles of inclination wherein the error is not triggered unless the distance error measurement is greater than the threshold, such as 1 mm or 2 mm. In another example, a threshold error angle can be set between the measured first and second angles of inclination (e.g., 0.5 degrees).

The one or more controllers 24, 74 can be configured to generate feedback to a user in response to detection of the error. In one example, the audible, visual, and/or haptic feedback is provided through the tracker assembly 44, 46, in the manner described above. Additionally, or alternatively, the feedback can be provided through one or both sensors 62, 62′ e.g., using an audible alarm or through the navigation system 12, e.g., using a visual or audiovisual alert, message or notification provided by the display unit(s) 32, 34.

Having described the solution, several configurations of the tracker observation sensor 62′ are contemplated for which the solution can fully be implemented. In one example, the tracker observation sensor 62′ is coupled to the tracker assembly 44, 46 itself. Here, the tracker observation sensor 62′ is rigidly attached to the bone by virtue of the tracker assembly 44, 46 to which it is attached. The tracker observation sensor 62′ can be integrally coupled to the tracker assembly 44, 46 or detachably coupled (and removable from) the tracker assembly 44, 46. In this configuration, the tracker assembly would support both the tracker motion sensor 62 and the tracker observation sensor 62′. The tracker motion sensor 62 can be coupled to the attachment portion, to the stem, or to any other component of the tracker assembly 44, 46 (including in a fixed relationship to at least one optical marker 48). Meanwhile, the tracker observation sensor 62′ can be coupled to any different component of the tracker assembly 44, 46 other than component to which the tracker motion sensor 62′ is attached (e.g., attachment, stem, tracking head, or fixed relationship to at the least one optical marker 48). The two tracker components respectively supporting the two sensors 62, 62′ should be moveable relative to each other to optimize the robustness of error detection. In this configuration, either sensor 62, 62′ can detect whether the component of the tracker assembly (to which it is attached) has moved relative to the other, thereby indicating the tracker assembly 44, 46 has experienced a distortion from its original configuration.

In another implementation, the tracker observation sensor 62′ is rigidly attached to the bone at a second location spaced apart and different from the first location (at which the “first” tracker assembly 44 and tracker motion sensor 62 are attached). In this configuration, by being spaced apart and separated from the first tracker assembly 44, the tracker observation sensor 62′ can detect whether any part of the first tracker assembly 44, including at least one optical marker 48, has moved relative to the bone.

In one example, the tracker observation sensor 62′ is coupled to a second tracker assembly (e.g., 46) is rigidly attached to the bone at the second location. The second tracker assembly 46 can include any of the described tracking configurations or modalities (e.g., optical tracking or the like). The tracker observation sensor 62′ can be integrally or removably coupled to any component of the second tracker assembly 46, including directly adjacent the optical marker(s). In such instances, the tracked pose of the optical marker(s) of the second tracker assembly 46 can optionally be combined to determine the baseline relationship. However, this need not be required as the relationship between the second measured angle of inclination can be fixed or moveable relative to the tracked pose of the optical marker(s) of the second tracker assembly 46.

In another example, the tracker observation sensor 62′ is detachably coupled to or integrated with a tracker observation device rigidly attached to the bone at the second location. The tracker observation device may be dedicated device specifically intended for the purpose of remotely monitoring the first tracker assembly 44. The tracker observation device may be used in addition to, or instead of, the second tracker assembly 46. In one configuration, the tracker observation device comprises a body that supports the tracker observation sensor 62′. The body of the tracker observation device supports an attachment that is configured to be mounted to the bone at the second location. The attachment can be a bone plate, a bone pin, a bone fastener, a clamp, limb strap, or the like. The body also supports the communication device of the tracker observation sensor 62′ to remotely transmit the second measured angle of inclination to the one or more controller 24, 74.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the examples is described above as having certain features, any one or more of those features described with respect to any example of the disclosure can be implemented in and/or combined with features of any of the other examples, even if that combination is not explicitly described. In other words, the described examples are not mutually exclusive, and permutations of one or more examples with one another remain within the scope of this disclosure.

Spatial and functional relationships between elements (for example, between controllers, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements.

As used herein, the phrase at least one of A, B, and C should be construed to mean a logical (A OR B OR C), using a non-exclusive logical OR, and should not be construed to mean “at least one of A, at least one of B, and at least one of C.” The term subset does not necessarily require a proper subset. In other words, a first subset of a first set may be coextensive with (equal to) the first set.

In the FIGS., the direction of an arrow, as indicated by the arrowhead, generally demonstrates the flow of information (such as data or instructions) that is of interest to the illustration. For example, when element A and element B exchange a variety of information, but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “controller” or “module” may be replaced with the term “circuit.” The term “controller” may refer to, be part of, or include: at least one Application Specific Integrated Circuit (ASIC); at least one programmable system on a chip (PSoC); at least one digital, analog, or mixed analog/digital discrete circuit; at least one digital, analog, or mixed analog/digital integrated circuit; at least one combinational logic circuit; at least one field programmable gate array (FPGA); at least one processor (shared, dedicated, or group) that executes code; at least one memory circuit (shared, dedicated, or group) that stores code executed by the at least one processor; other suitable hardware components that provide the described functionality; or a combination of some or all of the above, such as in a system-on-chip.

The controller may include one or more interface circuits with one or more transceivers, such as radio frequency (RF) or optical based transceivers (e.g., infrared (IR)). In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The controller may communicate with other controllers using the interface circuit(s). Although the controller may be depicted in the present disclosure as logically communicating directly with other controllers, in various implementations the controller may actually communicate via a communications system. The communications system may include physical and/or virtual networking equipment such as hubs, switches, routers, gateways, and transceivers. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the controller may be distributed among multiple controllers that are connected via the communications system. For example, multiple controllers may implement the same functionality distributed by a load balancing system. In a further example, the functionality of the controller may be split between a server (also known as remote, or cloud) controller and a client (or, user) controller.

Some or all hardware features of a controller may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a controller may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. The term shared processor circuit encompasses a single processor circuit that executes some or all code from multiple controllers. The term group processor circuit encompasses a processor circuit that, in combination with additional processor circuits, executes some or all code from one or more controllers. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple controllers. The term group memory circuit encompasses a memory circuit that, in combination with additional memories, stores some or all code from one or more controllers.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a computer to execute one or more particular functions embodied in computer programs. The functional blocks and flowchart elements described above may serve as software specifications, which can be translated into the computer programs by the routine work of a skilled technician or programmer.

The computer programs may include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A surgical navigation system for tracking a bone during a medical procedure, comprising:
a first tracker assembly having a first single optical marker and a motion sensor, the first tracker assembly being coupled to the bone at a first location;
a second tracker assembly having a second single optical marker, the second tracker assembly being coupled to the bone at a second location different from the first location;
a localizer configured to optically track positions of the first and second single optical markers in a coordinate system of the localizer; and
one or more controllers configured to:
determine a pose of a tracker coordinate system associated with the first and second tracker assemblies in the localizer coordinate system according to three positional degrees of freedom and two rotational degrees of freedom based on the tracked positions of the first and second single optical markers;
utilize measurements from the motion sensor to determine an angle of inclination with respect to gravity;
determine an orientation of the tracker coordinate system according to a further rotational degree of freedom defined about a virtual line extending between the first and second single optical markers based on the angle of inclination; and
determine a pose of the bone in the localizer coordinate system according to six degrees of freedom based on the determined pose and orientation of the tracker coordinate system.

2. The surgical navigation system of claim 1, wherein the motion sensor is configured to measure movement relative to an axis substantially perpendicular with the virtual line extending between the first and second single optical markers, and the angle of inclination with respective to gravity is defined as an angle of inclination of the axis with respect to gravity.

3. The surgical navigation system of claim 1, wherein the motion sensor is configured to measure movement relative to an axis, and the angle of inclination with respect to gravity is defined as an angle of inclination of the axis with respect to a plane normal to a gravity vector.

4. The surgical navigation system of claim 1, wherein the motion sensor is configured to measure movement relative to an axis, the angle of inclination with respect to gravity is defined as an angle of inclination of the axis with respect to gravity, and the one or more controllers are configured to:
determine an angle between the axis and a plane normal to the virtual line extending between the first and second single optical markers; and
determine the orientation of the tracker coordinate system according to the further rotational degree of freedom defined about the virtual line extending between the first and second single optical markers based on the determined angle of inclination and the angle between the axis and the plane normal to the virtual line.

5. The surgical navigation system of claim 1, wherein the motion sensor is defined as an accelerometer.

6. The surgical navigation system of claim 1, wherein first tracker assembly and the second tracker assembly each does not include a magnetometer and does not include a gyroscope.

7. The surgical navigation system of claim 1, wherein the first and second tracker assemblies are removably coupleable to first and second pedicle screws inserted into the bone, respectively.

8. The surgical navigation system of claim 1, wherein the bone is a vertebra.

9. The surgical navigation system of claim 1, wherein the one or more controllers are configured to:
determine an initial pose of the tracker coordinate system in the localizer coordinate system according to six degrees of freedom;
utilize measurements from the motion sensor that correspond to the initial pose to obtain an initial angle of inclination relative to gravity;
determine the pose of the tracker coordinate system in the localizer coordinate system according to three positional degrees of freedom and two rotational degrees of freedom based on the tracked positions of the first and second single optical markers and the initial pose; and
determine the orientation of the tracker coordinate system according to the further rotational degree of freedom based on the determined angle of inclination and the initial angle of inclination.

10. The surgical navigation system of claim 9, wherein the one or more controllers are configured to:
calculate a first vector connecting the first and second single optical markers in the localizer coordinate system based on the initial pose;
calculate a second vector connecting the first and second single optical markers in the localizer coordinate system based on the tracked positions of the first and second single optical markers;
calculate a rotational matrix from the first and second vectors; and
determine the pose of the tracker coordinate system in the localizer coordinate system according to the two rotational degrees of freedom based on the rotational matrix.

11. The surgical navigation system of claim 9, wherein the one or more controllers are configured to:
calculate a rotational matrix from the initial and determined angles of inclination; and
determine the orientation of the tracker coordinate system according to the further rotational degree of freedom based on the rotational matrix calculated from the initial and determined angles of inclination.

12. The surgical navigation system of claim 9, comprising a third tracker assembly including at least one optical marker detectable by the localizer, wherein at least one of the first and second tracker assemblies includes a feature for temporary contact by the third tracker assembly for determining the initial pose of the tracker coordinate system in the localizer coordinate system according to six degrees of freedom.

13. The surgical navigation system of claim 9, comprising a second motion sensor moveable with the localizer, wherein the one or more controllers are configured to:
utilize measurements from the second motion sensor that correspond to the initial pose to obtain a second initial angle of inclination with respect to gravity; and
determine the orientation of the tracker coordinate system according to the further rotational degree of freedom based on the determined angle of inclination, the initial angle of inclination, and the second initial angle of inclination.

14. The surgical navigation system of claim 1, wherein the one or more controllers are configured to monitor measurements from the motion sensor to determine whether either of the first or second tracker assemblies has moved relative to the bone.

15. A surgical system including a robotic manipulator and the surgical navigation system of claim 1, wherein the one or more controllers are configured to control movement of the robotic manipulator relative to the bone during the medical procedure based on the determined pose of the bone.

16. A method for tracking a bone during a medical procedure using a first tracker assembly including a first single optical marker and a motion sensor, and a second tracker assembly including a second single optical marker, the method comprising:
securing the first tracker assembly to the bone at a first location and securing the second tracker assembly to the bone at a second location different from the first location;
tracking optically, with a localizer, positions of the first and second single optical markers in a coordinate system of the localizer;
determining, by one or more controllers, a pose of a tracker coordinate system associated with the first and second tracker assemblies in the localizer coordinate system according to three positional degrees of freedom and two rotational degrees of freedom based on the tracked positions of the first and second single optical markers;
utilizing, by the one or more controllers, measurements from the motion sensor to determine an angle of inclination with respect to gravity;
determining, by the one or more controllers, an orientation of the tracker coordinate system according to a further rotational degree of freedom defined about a virtual line extending between the first and second single optical markers based on the angle of inclination; and
determining, by the one or more controllers, a pose of the bone in the localizer coordinate system according to six degrees of freedom based on the determined pose and orientation of the tracker coordinate system.

17. The method of any claim 16, wherein securing the first and second tracker assemblies to the bone comprises removably securing the first and second tracker assemblies to first and second pedicle screws inserted into the bone, respectively.

18. The method of claim 16, comprising:
determining an initial pose of the tracker coordinate system in the localizer coordinate system according to six degrees of freedom;
utilizing measurements from the motion sensor that correspond to the initial pose to obtain an initial angle of inclination relative to gravity;
determining the pose of the tracker coordinate system in the localizer coordinate system according to three positional degrees of freedom and two rotational degrees of freedom based on the tracked positions of the first and second single optical markers and the initial pose; and
determining the orientation of the tracker coordinate system according to the further rotational degree of freedom based on the determined angle of inclination and the initial angle of inclination.

19. A hybrid tracker for tracking a bone in six degrees of freedom, the hybrid tracker comprising:
a first tracker assembly having a first single optical marker and a motion sensor, the first tracker assembly being coupled to the bone at a first location; and
a second tracker assembly having a second single optical marker, the second tracker assembly being coupled to the bone at a second location different from the first location;
wherein motion sensor is configured to produce measurements indicative of an angle of inclination with respect to gravity and to measure movement relative to an axis substantially perpendicular with a virtual line extending between the first and second single optical markers.

20. The hybrid tracker of claim 19, wherein the bone is a vertebra and wherein:
the first tracker assembly comprises a first pedicle screw configured to be inserted into the vertebra, and the first single optical marker is coupled the first pedicle screw; and
the second tracker assembly comprises a second pedicle screw configured to be inserted into the vertebra, and the second single optical marker is coupled to the second pedicle screw.
